(19) United States
(12) Patent Application Publication
(10) Pub. No.: US 2023/0039415 A1
(43) Pub. Date: Feb. 9, 2023

(54) VITAL SIGN DETECTION DEVICE, VEHICLE INCLUDING THE SAME IN SEAT, AND VITAL SIGN DETECTION METHOD

(71) Applicant: Murata Manufacturing Co., Ltd., Nagaokakyo-shi (JP)

(72) Inventor: Daichi UEKI, Nagaokakyo-shi (JP)

(73) Assignee: Murata Manufacturing Co., Ltd., Nagaokakyo-shi (JP)

(21) Appl. No.: 17/939,973

(22) Filed: Sep. 8, 2022

Related U.S. Application Data
(63) Continuation of application No. PCT/JP2021/005038, filed on Feb. 10, 2021.

(30) Foreign Application Priority Data
Mar. 11, 2020 (JP) 2020-042460
Aug. 31, 2020 (JP) 2020-146416

(51) Int. Cl. A61B 5/05; A61B 5/00

(52) U.S. Cl. CPC A61B 5/05; A61B 5/6893; A61B 5/6844; A61B 5/7246; B60N 2/002

(57) ABSTRACT

A vital sign detection device controls directivities of radio waves A and B toward an irradiation region of a subject to determine the vital signs of the subject. The first directivity is where the vital signs easily appear and the second directivity is where the vital signs are less likely to appear. Noise is reduced by taking a difference between information about a distance to the subject calculated on the basis of the radio wave A having the first directivity and information about a distance to the subject calculated on the basis of the radio wave B having the second directivity received by the receiver.

### CROSS REFERENCE TO RELATED APPLICATION

The present application is a continuation application of International Patent Application No. PCT/JP2021/005038, filed Feb. 10, 2021, which claims priority to Japanese patent application No. 2020-042460, filed Mar. 11, 2020, and Japanese patent application No. 2020-146416, filed Aug. 31, 2020, the entire contents of each of which being incorporated herein by reference.

### TECHNICAL FIELD

The present disclosure relates to a vital sign detection device for detecting the vital signs of a subject, a vehicle including the vital sign detection device in a seat, a vital sign detection method, and a vital sign detection computer program product.

### BACKGROUND ART

For example, Patent Document 1 discloses a vital sign detection device and a vital sign detection method. In this vital sign detection device and this vital sign detection method, a specifying unit specifies, as a specific part suitable for the detection of vital signs, a measurement part where a frequency analysis result regarding the reflected wave of an electromagnetic wave applied to a subject indicates frequency characteristics in a physical state in which the vital signs of the subject strongly appear. A control unit controls the scanning of an electromagnetic wave irradiation region such that the specific part specified by the specifying unit is irradiated with an electromagnetic wave. A detection unit detects the vital signs of the subject on the basis of the analysis result of a reflected wave received at the specific part specified by the specifying unit.

For example, examples of a vital sign detection device include a biological sensor disclosed in Patent Document 2. This biological sensor is a non-contact type biological sensor for detecting human biological information by an electromagnetic wave, and two sets of the biological sensors are provided in a seat on which a person sits. The biological sensors in each set are a first sensor and a second sensor that emit electromagnetic waves of different frequencies to a person and are disposed next to each other. One of the first sensor and the second sensor is used for the detection of biological information including a noise element, and the other one of them is used for the detection of a noise element. By taking the difference, which corresponds to the noise element, between them, the biological information of a subject is extracted.

### CITATION LIST

### Patent Document

Patent Document 1: Japanese Unexamined Patent Application Publication No. 2019-115464
Patent Document 2: Japanese Unexamined Patent Application Publication No. 2019-180451

### SUMMARY

### Technical Problems

However, as recognized by the present inventor, one example issue with the above vital sign detection device and the above vital sign detection method in the related art disclosed in Patent Document 1 it that they specify a measurement part where the vital signs of a subject strongly appear and thus they simply detect the vital signs of the subject from the measurement part. Accordingly, the detected vital signs of the subject include noise, such as the body movement of the subject, superimposed on the measurement part where vital signs strongly appear, and the vital signs of the subject cannot be accurately detected, due to the presence of the superimposed noise.

In the biological sensor in the related art disclosed in Patent Document 2, the vital signs of a subject are accurately extracted by subtracting a noise element detected by one of a first sensor and a second sensor, which are biological sensors, from biological information including the noise element detected by the other one of them. However, as recognized by the present inventor, if the size and orientation of the subject change, the part of the subject to which the first sensor and the second sensor apply respective electromagnetic waves also change. Thus, a case may arise where electromagnetic waves are not applied to a part where biological information easily appears. In such a case, the vital signs of the subject cannot be accurately extracted even by taking the difference between pieces of information detected by the respective sensors. Furthermore, the first sensor and the second sensor need to be disposed next to each other. Accordingly, the biological sensors occupy a large space in, for example, a seat in a vehicle in which the biological sensors are provided. Places of installation of the biological sensors are therefore limited in, for example, a seat in a vehicle, and the degree of freedom in design decreases. Furthermore, the use of a plurality of sensors including the first sensors and the second sensors increases the cost of a device.

### Solutions to Problems

The present disclosure has been made to solve the above-identified and other problems with conventional approaches.

A vital sign detection device includes a transmitter configured to emit electromagnetic waves toward a subject; a scan controller configured to change a directivity of transmissions from the transmitter so as to scan an irradiation region of the subject with the electromagnetic waves; a receiver configured to receive a plurality of returned electromagnetic waves with different directivities after having reflected off the subject; and a vital sign extraction circuitry configured to extract vital signs of the subject using a difference between distance information of the subject calculated based on a particular electromagnetic wave that shows vital signs of the subject and has a highest signal intensity of the plurality of the electromagnetic waves received by the receiver and distance information of the subject calculated based on another electromagnetic wave that shows characteristics other than vital signs of the subject and has a highest signal intensity of the plurality of electromagnetic waves received by the receiver that show characteristics other than the vital signs of the subject.

A vital sign detection method according to the present disclosure includes, directing with a scan controller an electromagnetic wave emitted from a transmitter to scan an irradiation region of a subject by changing a directivity of transmissions from the transmitter; receiving a plurality of reflected electromagnetic waves having different directivities after reflection off the subject; extracting, with a processor, vital signs of the subject using a difference between distance information of the subject calculated based on a particular electromagnetic wave that shows vital signs of the subject and has the highest signal intensity of a plurality of electromagnetic waves received by the receiver and distance information of the subject calculated based on another electromagnetic wave that shows characteristics other than vital signs of the subject and has the highest signal intensity of the plurality of electromagnetic waves; comparing a size of vital signs of the subject extracted in the extracting with a predetermined threshold value; and performing re-extraction under a condition the size of vital signs of the subject extracted in the vital sign extraction step does not exceed the predetermined threshold value as a result of the comparing, and repeatedly performing the directing, receiving, and extracting.

With this configuration, the directivities of electromagnetic waves to be emitted from the single irradiation unit to the subject are changed to the first directivity and the second directivity and the electromagnetic wave having the first directivity and the electromagnetic wave having the second directivity are applied from the irradiation unit to the irradiation region where the vital signs of the subject easily appear and the irradiation region where the vital signs of the subject are less likely to appear, respectively by control processing that the control unit performs upon the scan unit. Noise included in the vital signs of the subject is removed and the vital signs of the subject are accurately extracted by causing the vital sign extraction unit to take a difference between information about the distance from the vital sign detection device to the electromagnetic wave irradiation point of the subject calculated on the basis of an electromagnetic wave having the first directivity that has been reflected from the subject and received by the reception unit and information about the distance from the vital sign detection device to the electromagnetic wave irradiation point of the subject calculated on the basis of an electromagnetic wave having the second directivity that has been reflected from the subject and received by the reception unit.

Even if the size and orientation of the subject change, the directivities of electromagnetic waves to be emitted from the irradiation unit to the subject are changed to the first directivity of an electromagnetic wave with which an electromagnetic wave is applied to the irradiation region where the vital signs of the subject easily appear and the second directivity of an electromagnetic wave with which an electromagnetic wave is applied to the irradiation region where the vital signs of the subject are less likely to appear by control processing that the control unit performs upon the scan unit. Accordingly, even if the size and orientation of the subject change, electromagnetic waves are emitted from the irradiation unit to the subject and applied to the irradiation region where the vital signs of the subject easily appear and the irradiation region where the vital signs of the subject are less likely to appear. As a result, noise included in the vital signs of the subject is removed and only the vital signs of the subject are accurately extracted by the vital sign extraction unit.

Since the vital signs of the subject are accurately extracted using the single irradiation unit as above, space occupied by the vital sign detection device at an installation location can be reduced, the scope of selection of an installation location of the vital sign detection device can be broadened, and the degree of freedom in design increases. In addition, the cost of the vital sign detection device can be reduced.

A vital sign detection device according to the present disclosure includes,

an irradiation unit configured to emit an electromagnetic wave to a subject,

a scan unit configured to scan an irradiation region of an electromagnetic wave to be applied to the subject by changing a directivity of an electromagnetic wave to be emitted from the irradiation unit,

a reception unit configured to receive a plurality of electromagnetic waves having different directivities that have hit against and been reflected from the subject, and

a vital sign extraction unit configured to extract vital signs of the subject using a difference between distance information of the subject calculated based on an electromagnetic wave that shows vital signs of the subject and has the highest signal intensity of a plurality of electromagnetic waves received by the reception unit and distance information of the subject calculated based on an electromagnetic wave that shows characteristics other than vital signs of the subject and has the highest signal intensity of the plurality of electromagnetic waves.

A vital sign detection method according to the present disclosure includes,

an electromagnetic wave scan step of causing a scan unit to scan an irradiation region of an electromagnetic wave for a subject by changing a directivity of an electromagnetic wave to be applied from an irradiation unit to the subject,

an electromagnetic wave reception step of causing a reception unit to receive a plurality of electromagnetic waves having different directivities that have been subjected to scanning in the electromagnetic wave scan step and that have hit against and been reflected from the subject,

a vital sign extraction step of extracting vital signs of the subject using a difference between distance information of the subject calculated based on an electromagnetic wave that shows vital signs of the subject and has the highest signal intensity of a plurality of electromagnetic waves received by the reception unit and distance information of the subject calculated based on an electromagnetic wave that shows characteristics other than vital signs of the subject and has the highest signal intensity of the plurality of electromagnetic waves,

a comparison step of comparing a size of vital signs of the subject extracted in the vital sign extraction step with a predetermined threshold value, and

a vital sign re-extraction step of, when the size of vital signs of the subject extracted in the vital sign extraction step does not exceed the predetermined threshold value as a result of the comparison step, repeatedly performing the electromagnetic wave scan step, the electromagnetic wave reception step, and the vital sign extraction step.

In this configuration, electromagnetic waves to be emitted from the single irradiation unit to the subject are caused to have a plurality of directivities and applied from the irradiation unit. The electromagnetic waves applied from the irradiation unit are received by the reception unit as electromagnetic waves having a plurality of directivities. Noise included in the vital signs of the subject is removed and the vital signs of the subject are accurately extracted by causing the vital sign extraction unit to take a difference between the distance information of the subject calculated on the basis of an electromagnetic wave that shows the vital signs of the subject and has the highest signal intensity of a plurality of electromagnetic waves received by the reception unit and the distance information of the subject calculated on the basis of an electromagnetic wave that shows characteristics other than the vital signs of the subject and has the highest signal intensity of the multiple electromagnetic waves.

Even if the size and orientation of the subject change, electromagnetic waves having a plurality of new directivities are received by the reception unit by causing the scan unit to change the directivities of electromagnetic waves to be emitted from the irradiation unit to the subject. Noise included in the vital signs of the subject is removed and the new vital signs of the subject are accurately extracted by causing the vital sign extraction unit to take a difference between the distance information of the subject calculated on the basis of an electromagnetic wave that shows the vital signs of the subject and has the highest signal intensity of a plurality of new electromagnetic waves received by the reception unit and the distance information of the subject calculated on the basis of an electromagnetic wave that shows characteristics other than the vital signs of the subject and has the highest signal intensity of the multiple new electromagnetic waves. Thus, the vital signs of a subject are accurately extracted even if the size and orientation of the subject change.

Since the vital signs of the subject are accurately extracted using the single irradiation unit as above also in this configuration, space occupied by the vital sign detection device at an installation location can be reduced, the scope of selection of an installation location of the vital sign detection device can be broadened, and the degree of freedom in design increases. In addition, the cost of the vital sign detection device can be reduced.

### Advantageous Effects of the Disclosure

According to the present disclosure, one non-exclusive advantage is that there can be provided a vital sign detection device and a vital sign detection method with which noise included in the vital signs of a subject can be removed and only the vital signs of the subject can always be accurately extracted even if the size and orientation of the subject change, and the increase in the degree of freedom in design and cost reduction can be achieved.

### BRIEF DESCRIPTION OF DRAWINGS

FIG. 1 is a schematic side view of a seat on which a subject sits in a vehicle to which a vital sign detection device and a vital sign detection method according to a first embodiment of the present disclosure are applied.

FIG. 2 is a block diagram illustrating the schematic configuration of a vital sign detection device according to the first embodiment of the present disclosure.

FIG. 3 is a flowchart illustrating a rough process in a vital sign detection method according to the first embodiment of the present disclosure.

FIG. 4 is a flowchart illustrating a detailed process in the vital sign detection method according to the first embodiment of the present disclosure.

FIG. 5 is a graph describing the detection of vital signs of a subject from the displacement of a body surface of the subject calculated by a vital sign detection device and a vital sign detection method according to the first embodiment of the present disclosure.

FIG. 6 is a schematic side view of a seat on which a subject sits in a vehicle to which a vital sign detection device and a vital sign detection method according to a second embodiment of the present disclosure are applied.

FIG. 7 is a flowchart schematically illustrating a vital sign detection method for which a vital sign detection device according to a fourth embodiment of the present disclosure is used.

FIGS. 8A and 8B FIG. 8A is a perspective view of the interior of a car in which vital sign detection devices according to an embodiment of the present disclosure are disposed at respective positions, and FIG. 8B is a perspective view of the interior of a hospital room in which vital sign detection devices according to an embodiment of the present disclosure are disposed at respective positions.

### DESCRIPTION OF EMBODIMENTS

Next, embodiments of a vital sign detection device according to the present disclosure, a vehicle including the vital sign detection device in a seat, and a vital sign detection method will be described.

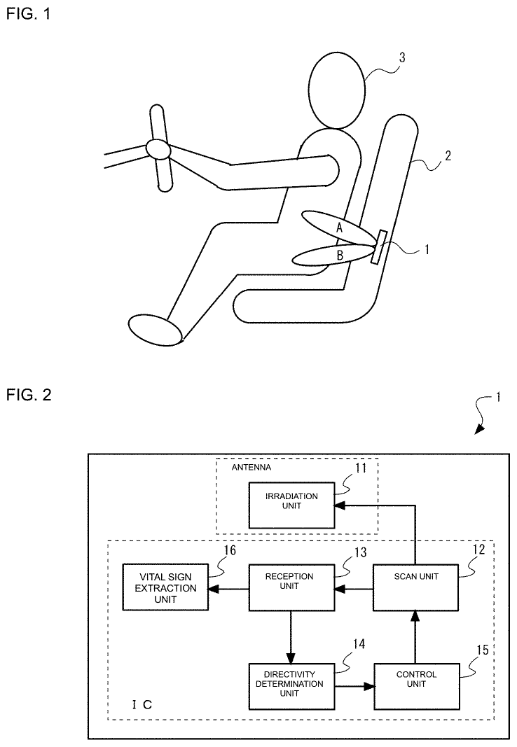

FIG. 1 is a schematic side view of a seat 2 on which a subject 3 sits in a vehicle to which a vital sign detection device 1 and a vital sign detection method according to a first embodiment of the present disclosure are applied as a driver monitoring system (DMS) and. The vital sign detection device 1 and a vital sign detection method according to this embodiment detect, as vital signs, the body surface displacement of the subject 3 driving a vehicle. The body surface displacement serves as an indicator of vital signs, such as the heart rate, heart rate variability, respiration rate, and depth of breathing of the subject 3, that is, the variation in distance from the vital sign detection device 1 to an electromagnetic wave irradiation point on the body surface of the subject 3.

The vital sign detection device 1 according to the first embodiment is disposed in the seat 2 (e.g., perhaps disposed in the fabric, or outer surface material, such as leather, of the seat, or in an interior portion of the seat) and has the configuration schematically illustrated in the block diagram in FIG. 2. The vital sign detection device 1 includes an irradiation unit 11 (also referred to as a transmitter, electromagnetic (EM) wave transmitter, or transmitter circuitry), a scan unit 12 (also described as scan controller or scan control circuitry that controls a change of direction of the EM waves emitted from the irradiation unit 11 so as to scan across a region of the subject), a reception unit 13 (also referred to as a receiver or receive circuitry), a directivity determination unit 14 (or directivity determination circuitry implemented separately, or by the controller, such as a software-based processor executed in the controller), a control unit 15 (or a controller, or control circuitry), and a vital sign extraction unit 16 (or vital sign extraction circuitry implemented separately, or by the controller, such as a software-based processor executed in the controller). Each of these “units” may be implemented as hardware, firmware, and/or software control processing executed by a microcomputer (an example of a controller), the hardware configuration of an electronic circuit, or both of them. In general, the irradiation unit 11 is configured as an antenna, and the scan unit 12, the reception unit 13, the directivity determination unit 14, the control unit 15, and the vital sign extraction unit 16 are configured as an IC (integrated circuit), or circuitry (one or more ICs, microcomputers, and/or computers). A non-limiting example of a processing circuitry that may be programmed according to the present teachings to implement the “units” described above is found in U.S. Pat. No. 10,270,856, the entire contents of which is incorporated herein by reference.

The irradiation unit 11 includes a Doppler radar (or Doppler radar system) or an FMCW (frequency modulated continuous wave) radar (for FMCW system) including one or more transmission antennas and a transmitter that emits radio frequency (RF) EM waves from the transmission antennas. The units other than the vital sign extraction unit 16: the irradiation unit 11, the scan unit 12, the reception unit 13, the directivity determination unit 14, and the control unit 15, form the radar (or radar system). The antenna includes an electromagnetic lens or a patch antenna formed as a conductor pattern on a circuit board. Although an electromagnetic wave emitted by the irradiation unit 11 will be described as a radio wave in this non-limiting embodiment, other applicable examples include acoustic waves (ultrasonic waves) as well as electromagnetic waves outside of the RF spectrum, including for example light waves. The scan unit 12 scans an irradiation region with a radio wave (or more generally an EM wave) to be applied to the subject 3 by changing the directivity of a radio wave to be emitted from the irradiation unit 11. Accordingly, the irradiation unit 11 can emit an electromagnetic wave and change the directivity of an electromagnetic wave to be emitted.

The directivity of a radio wave to be emitted from the irradiation unit 11 can be changed by changing the phase and amplitude of a radio wave to be emitted from the irradiation unit 11 using an analog beamforming system or a digital beamforming system in which the phase and amplitude of a radio wave received by the reception unit 13 is controlled and calculated. In the analog beamforming system, the directivity of a radio wave to be emitted from the irradiation unit 11 is changed on the basis of information about the phase and amplitude of a radio wave that the irradiation unit 11 receives from the scan unit 12 or the control unit 15. In the digital beamforming system, a similar effect of changing the directivity of a radio wave to be emitted from the irradiation unit 11 can be obtained by causing the control unit 15 or the scan unit 12 to calculate the phase and amplitude of a radio wave received by the reception unit 13.

The reception unit 13 includes one or more reception antennas and receives a radio wave that has been subjected to scanning by the scan unit 12 and hit against and reflected from the subject 3. The directivity determination unit 14 determines, from a result of reception of radio waves in the reception unit 13, a first directivity of a radio wave with which a radio wave is applied to an irradiation region where the vital signs of the subject 3 easily appear and a second directivity of a radio wave with which a radio wave is applied to an irradiation region where the vital signs of the subject 3 are less likely to appear. In FIG. 1, a radio wave A having a first directivity and a radio wave B having a second directivity are illustrated. In this embodiment, the respective frequency band(s) of the radio wave A having the first directivity and the radio wave B having the second directivity are set to be the same.

The reception unit 13 includes a calculation portion that calculates, as the body surface displacement of the subject 3, a difference between information about the distance from the vital sign detection device 1 to the electromagnetic wave irradiation point of the subject 3 calculated on the basis of an electromagnetic wave having the first directivity that has been reflected from the subject 3 and received by the reception unit 13 and information about the distance from the vital sign detection device 1 to the electromagnetic wave irradiation point of the subject 3 calculated on the basis of an electromagnetic wave having the second directivity that has been reflected from the subject 3 and received by the reception unit 13. The directivity determination unit 14 includes a candidate determination portion (in this embodiment a software process implemented in a programmed controller, but may also be a dedicated circuit and or IC) that determines the irradiation direction of each of electromagnetic waves that are respective candidates for the first directivity and the second directivity and instructs the control unit 15 such that an electromagnetic wave to be emitted from the irradiation unit 11 has the determined irradiation direction and a direction determination portion to determine the first directivity and the second directivity and instruct the control unit 15 such that respective electromagnetic waves to be emitted from the irradiation unit 11 have the determined first directivity and the determined second directivity.

The candidate determination portion determines, for respective candidates for the first directivity and the second directivity, the irradiation range of an electromagnetic wave to be emitted from the irradiation unit 11 to the subject 3 and the size of a scan angle at which the irradiation range is scanned with an electromagnetic wave. For example, for a candidate for the first directivity, the irradiation range of an electromagnetic wave is set to ±30° with respect to the direction of the main lobe of RF energy emitted from the irradiation unit 11 and the scan angle of 1° is determined. In this case, irradiation and scanning are performed with an electromagnetic wave in, for example, the directions of +30°, +29°, +28°, . . . with respect to the direction of the main lobe of RF energy emitted from the irradiation unit 11. For a candidate for the second directivity, an irradiation range different from the irradiation range of an electromagnetic wave determined for a candidate for the first directivity and the size of a scan angle at which the irradiation range is scanned with an electromagnetic wave are determined. Subsequently, the candidate determination portion instructs the control unit 15 such that an electromagnetic wave is emitted from the irradiation unit 11 at the determined size of a scan angle in the determined irradiation range.

The direction determination portion (which in this non-limiting example is implemented as a software process executed on a programmable controller, but may also be implemented in one or more dedicated circuits) compares a plurality of reception waveforms of electromagnetic waves that have been applied in the irradiation direction determined as a candidate for the first directivity by the candidate determination portion, reflected off the subject 3, and a portion returned so as to be received by the reception unit 13 with a model waveform of an electromagnetic wave having the first directivity prepared in advance, calculate the degree of similarity between them, and determines the first directivity. Since electromagnetic waves having the first directivity have waveforms showing similar tendencies irrespective of who the subject 3 is (or the physical characteristics of the particular subject 3), the approximate waveform is set as a model waveform. The degree of similarity between the model waveform and each reception waveform is calculated using a waveform comparison method, such as the dynamic time warping method or the cross-correlation function. The directivity of an electromagnetic wave whose reception waveform has the highest degree of similarity to the model waveform is determined as the first directivity.

The control unit 15 controls scanning with a radio wave performed in response to the directivity determination unit 14 determining the first directivity and the second directivity and then performs control processing for setting the directivity of a radio wave to be applied to the subject 3 to the first directivity or the second directivity determined by the directivity determination unit 14. Under this control performed by the control unit 15 upon the scan unit 12, the application of the radio wave A from the irradiation unit 11 to the subject 3 and the application of the radio wave B from the irradiation unit 11 to the subject 3 are performed for a predetermined irradiation time period at predetermined time intervals. The predetermined irradiation time period is, for example, approximately 10 μsec to all the time, and the predetermined time interval is, for example, all the time to approximately 10 msec. When the predetermined time interval has elapsed, the radio waves A and B are continuously emitted from the irradiation unit 11. The radio waves A and B may be simultaneously emitted, or switching between the emissions of the radio waves A and B may be performed. The vital sign extraction unit 16 extracts the vital signs of the subject 3 on the basis of the difference, which is calculated by the calculation portion in the reception unit 13, between information about the distance from the vital sign detection device 1 to the electromagnetic wave irradiation point of the subject 3 calculated on the basis of the radio wave A having the first directivity that has been reflected from the subject 3 and received by the reception unit 13 and information about the distance from the vital sign detection device 1 to the electromagnetic wave irradiation point of the subject 3 calculated on the basis of the radio wave B having the second directivity that has been reflected from the subject 3 and received by the reception unit 13.

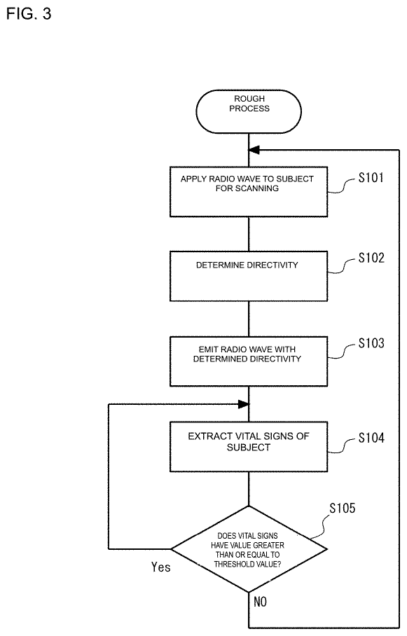

FIG. 3 is a flowchart illustrating a “rough” or high level process in a vital sign detection method according to the first embodiment.

For the detection of vital signs of the subject 3, first, a radio wave scanning step is performed in step (hereinafter abbreviated as “S”) 101 in FIG. 3. In this radio wave scanning step, the control unit 15 controls scan unit 12 to cause the scan unit 12 to scan the radio wave irradiation region of the subject 3 by changing the directivity of a radio wave to be emitted from the irradiation unit 11 to the subject 3. This scanning is performed using the irradiation range of an electromagnetic wave emitted from the irradiation unit 11 to the subject 3 and the size of a scan angle at which the irradiation range is scanned with an electromagnetic wave which are determined by the candidate determination portion in the directivity determination unit 14. Subsequently, in a directivity determination step in S102, the direction determination portion in the directivity determination unit 14 determines the first directivity of the radio wave A to be applied to an irradiation region where the vital signs of the subject 3 easily appear and the second directivity of the radio wave B to be applied to an irradiation region where the vital signs of the subject 3 are less likely to appear. This determination of a directivity is performed in such a manner that the degree of a similarity between the waveform of a radio wave received and a model waveform is calculated on the basis of a result of reception of radio waves that have been subjected to scanning in the radio wave scanning step in S101, impact and reflect from the subject 3, and received by the reception unit 13.

Subsequently, in a radio wave irradiation step in S103, the radio wave A having the first directivity determined in the above directivity determination step and the radio wave B having the second directivity determined in the above directivity determination step are emitted from the irradiation unit 11 to the subject 3 for the above-described predetermined irradiation time period at the above-described predetermined time intervals to irradiate the irradiation region where the vital signs of the subject 3 easily appear and the irradiation region where the vital signs of the subject 3 are less likely to appear. Subsequently, in a vital sign extraction step in S104, the body surface displacement of the subject 3 is detected and the vital signs of the subject 3 are extracted on the basis of the difference between information about the distance to the subject 3 calculated from the radio wave A having the first directivity that has been reflected from the subject 3 and received by the reception unit 13 and information about the distance to the subject 3 calculated from the radio wave B having the second directivity that has been reflected from the subject 3 and received by the reception unit 13.

Subsequently, in a comparison step in S105, the size of vital signs of the subject 3 extracted in the above vital sign extraction step is compared with a predetermined threshold value. When the size of vital signs of the subject 3 is greater than or equal to the threshold value and a result of the comparison in S105 is Yes, the process returns to S104 and the processing of S104 is repeated. On the other hand, when the size of vital signs of the subject 3 does not exceed the threshold value, the process returns to S101 and the radio wave scanning step in S101, the directivity determination step in S102, the radio wave irradiation step in S103, and the vital sign extraction step in S104 are repeated. Thus, a vital sign re-extraction step is performed. In the vital sign re-extraction step, the irradiation range of an electromagnetic wave emitted from the irradiation unit 11 to the subject 3 is scanned at all the scan angles determined by the candidate determination portion in the directivity determination unit 14.

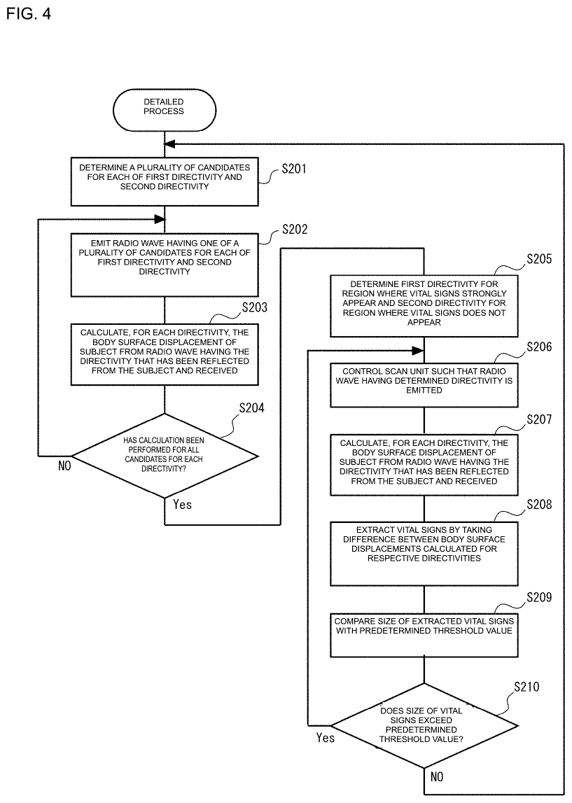

FIG. 4 is a flowchart illustrating a more detailed process in the vital sign detection method according to the first embodiment of the present disclosure.

For the detection of vital signs of the subject 3, more specifically, S201 in FIG. 4 is performed first. In S201, the above-described candidate determination portion in the directivity determination unit 14 determines a plurality of candidates for the first directivity of the radio wave A to be applied to the irradiation region where the vital signs of the subject 3 easily appear and the second directivity of the radio wave B to be applied to the irradiation region where the vital signs of the subject 3 are less likely to appear. A candidate for the first directivity and a candidate for the second directivity may be independently determined, or a candidate for the combination of the first directivity and the second directivity may be determined.

Subsequently, in S202, the control unit 15 controls the scan unit 12 to cause the irradiation unit 11 to emit a radio wave to the subject 3 with the directivity of one of the multiple candidates for the first directivity and the second directivity which is determined by the above-described candidate determination portion. Subsequently, in S203, the reception unit 13 calculates, for each directivity, the body surface displacement of the subject 3, that is, the change in distance from the vital sign detection device 1 to the electromagnetic wave irradiation point of the subject 3, from the radio wave A having the first directivity and the radio wave B having the second directivity that have been reflected from the subject 3 and received by the reception unit 13.

Subsequently, in S204, it is determined whether the body surface displacement of the subject 3 has been calculated for all of the multiple candidates for the respective directivities determined in S201. When the body surface displacement of the subject 3 has yet to be calculated for all of the multiple candidates for the respective directivities and a result of the determination in S204 is No, the process returns to S202 and the pieces of processing of S202 and S203 are repeated. By this looped process, the body surface displacement of the subject 3 is calculated for all of the multiple candidates for the respective directivities.

When the body surface displacement of the subject 3 has been calculated for all of the multiple candidates for the respective directivities and a result of the determination in S204 is Yes, the processing of S205 is performed. In S205, the radio wave A with which the body surface displacement having vital sign characteristics is obtained and which has the highest signal intensity of the radio waves A that are candidates for the first directivity for which the respective body surface displacements of the subject 3 have been calculated, that is, the radio wave A whose waveform has the highest degree of similarity to a model waveform, is selected. The directivity determination unit 14 determines that the directivity of the selected radio wave A is the first directivity with which the radio wave A is applied to the irradiation region where the vital signs of the subject 3 easily appear. The radio wave B is selected with which the body surface displacement having noise characteristics such as the body movement of the subject 3 and an automotive vibration is obtained and which has the highest signal intensity of the radio waves B that are candidates for the second directivity for which the respective body surface displacements of the subject 3 have been calculated. The directivity determination unit 14 determines that the directivity of the selected radio wave B is the second directivity with which the radio wave B is applied to the irradiation region where the vital signs of the subject 3 are less likely to appear.

Subsequently, in S206, the control unit 15 controls the scan unit 12 to cause the irradiation unit 11 to emit the radio wave A having the first directivity determined as above and the radio wave B having the second directivity determined as above to the subject 3. Subsequently, in S207, the reception unit 13 calculates, for each directivity, the body surface displacement of the subject 3 from each of the radio wave A having the first directivity and the radio wave B having the second directivity that have been reflected from the subject 3 and received by the reception unit 13.

It is considered that the body surface displacement of the subject 3 calculated from the radio wave A having the first directivity that has been applied to the irradiation region where the vital signs of the subject 3 easily appear and received by the reception unit 13 includes the vital signs of the subject 3 and noise such as the body movement of the subject 3 and an automotive vibration. It is also considered that the body surface displacement of the subject 3 calculated from the radio wave B having the second directivity that has been applied to the irradiation region where the vital signs of the subject 3 are less likely to appear and received by the reception unit 13 does not include the vital signs of the subject 3 and includes only noise such as the body movement of the subject 3 and an automotive vibration. Accordingly, in S208, the vital signs of the subject 3 are extracted by taking the difference between the body surface displacement of the subject 3 calculated for the first directivity and the body surface displacement of the subject 3 calculated for the second directivity.

This difference between the body surface displacements is calculated by subtracting the body surface displacement of the subject 3 calculated from the radio wave B having the second directivity received by the reception unit 13 from the body surface displacement of the subject 3 calculated from the radio wave A having the first directivity received by the reception unit 13. The difference between body surface displacements may be calculated by applying a Kalman filter or an adaptive filter to the body surface displacement of the subject 3 calculated from each of the radio waves A and B and blunting (or clipping) a surge signal that suddenly appears in a body surface displacement or by averaging body surface displacements in a certain time periods and performing the above subtraction. Alternatively, the difference between body surface displacements of the subject 3 may be calculated by separating a body movement, a vibration, and vital signs in each of the radio waves A and B by the audio source separation method, such as the independent component analysis or the independent vector analysis.

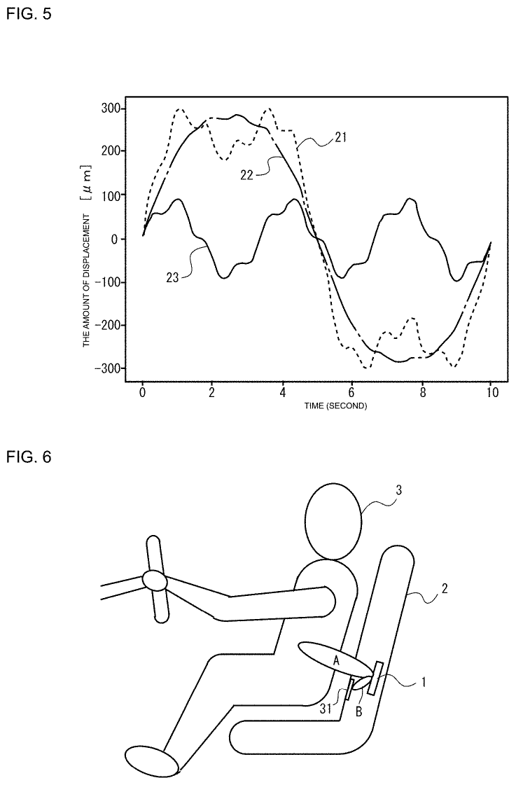

FIG. 5 is a graph representing time changes in the body surface displacement of the subject 3 calculated from the radio wave A having the first directivity determined in S205, the body surface displacement of the subject 3 calculated from the radio wave B having the second directivity determined in S205, and the difference between these body surface displacements. The horizontal axis represents time [second] and the vertical axis represents the amount of displacement of the body surface displacement of the subject 3 [μm] in the graph. The average of the amounts of displacement of the body surface displacement in a certain time segment is represented by 0. A characteristic line 21 indicated by a dotted line represents the change in the body surface displacement of the subject 3 calculated from the radio wave A having the first directivity over time, a characteristic line 22 indicated by a dot-and-dash line represents the change in the body surface displacement of the subject 3 calculated from the radio wave B having the second directivity over time, and a characteristic line 23 indicated by a solid line represents the change in the difference between these body surface displacements over time. Characteristics indicated by the characteristic line 23 indicating the difference represents the vital signs of the subject 3. A waveform from which vital signs are clearly observed like the waveform indicated by the characteristic line 23 is used as the above-described model waveform to be compared with each reception waveform received by the reception unit 13.

Subsequently, in S209, the size of vital signs of the subject 3 extracted in S208 in FIG. 4 is compared with a predetermined threshold value. As for the size of vital signs, the average of vital signs in a predetermined period is used. Subsequently, in S210, it is determined whether the extracted size of vital signs of the subject 3 is greater than or equal to a threshold value. When the extracted size of vital signs of the subject 3 is greater than or equal to the predetermined threshold value and a result of the determination in S210 is Yes, the process returns to S206 and pieces of processing of S206 to S209 are repeated. By this repeat of pieces of processing, the vital signs of the subject 3 are continuously extracted.

As the predetermined threshold value in S210, for example, a predetermined value of a power ratio SNR (signal-to-noise ratio) of a vital sign signal strength S to noise power N is used which is obtained in the frequency variation waveform of a vital sign signal strength obtained by performing the fast Fourier transform (FFT) upon vital signs represented by the characteristic line 23 in FIG. 5. When the SNR value is greater than a value determined in advance, it is determined in S210 that the size of vital signs of the subject 3 exceeds the predetermined threshold value.

In the case where the vital sign detection device 1 is formed by an FMCW radar, an IF (intermediate frequency) waveform is obtained by applying FFT to an IF signal of a reflected signal received by the reception unit 13 and a predetermined SNR value or a predetermined signal strength value in the frequency (distance) range of the IF waveform in which it is assumed that the subject 3 is present may be used as the predetermined threshold value in S210. In an FMCW radar, reflected power corresponding to a distance can be obtained by applying FFT to an IF signal. A signal having high reflected power in the frequency (distance) range in which it is assumed that the subject 3 is present is determined to be vital signs. Accordingly, when an IF waveform is obtained by applying FFT to an IF signal of a reflected signal received by the reception unit 13 and an SNR value or a signal strength value in a frequency (distance) range of the IF waveform in which it is assumed that the subject 3 is present is greater than a value determined in advance, it is determined in S210 that the size of vital signs of the subject 3 exceeds the predetermined threshold value.

On the other hand, when the extracted size of vital signs of the subject 3 does not exceed the predetermined threshold value and a result of the determination in S210 is No, the process returns to S201 and pieces of processing of S201 to S209 are repeated. By this repetition of processing, the new first directivity with which the radio wave A is applied to the new irradiation region where the vital signs of the subject 3 easily appear and the new second directivity with which the radio wave B is applied to the new irradiation region where the vital signs of the subject 3 are less likely to appear are determined, the radio waves A and B are applied to the respective new irradiation regions, and the new vital signs of the subject 3 are continuously extracted.

With the vital sign detection device 1 and a vital sign detection method according to the first embodiment, the directivities of the radio waves A and B to be emitted from the single irradiation unit 11 to the subject 3 are changed to the first directivity and the second directivity, respectively and the radio waves A and B are applied from the irradiation unit 11 to the irradiation region where the vital signs of the subject 3 easily appear and the irradiation region where the vital signs of the subject 3 are less likely to appear, respectively by the control that the control unit 15 performs upon the scan unit 12. Noise included in the vital signs of the subject 3 is removed and the vital signs of the subject 3 are accurately extracted by causing the vital sign extraction unit 16 to take the difference between information about the distance from the vital sign detection device 1 to the electromagnetic wave irradiation point of the subject 3 calculated on the basis of the radio wave A having the first directivity that has been reflected from the subject 3 and received by the reception unit 13 and information about the distance from the vital sign detection device 1 to the electromagnetic wave irradiation point of the subject 3 calculated on the basis of the radio wave B having the second directivity that has been reflected from the subject 3 and received by the reception unit 13.

Even if the size and orientation of the subject 3 change, the directivities of the radio waves A and B to be emitted from the irradiation unit 11 to the subject 3 are changed to the first directivity with which the radio wave A is applied to the irradiation region where the vital signs of the subject 3 easily appear and the second directivity with which the radio wave B is applied to the irradiation region where the vital signs of the subject 3 are less likely to appear, respectively by the control that the control unit 15 performs upon the scan unit 12. Accordingly, even if the size and orientation of the subject 3 change, the radio waves A and B are emitted from the irradiation unit 11 to the subject 3 and applied to the irradiation region where the vital signs of the subject 3 easily appear and the irradiation region where the vital signs of the subject 3 are less likely to appear, respectively, noise such as a body movement and a vibration included in the vital signs of the subject 3 is removed by the vital sign extraction unit 16, and only the vital signs of the subject 3 are accurately extracted.

Furthermore, since the vital signs of the subject 3 are accurately extracted using the single irradiation unit 11 in the vital sign detection device 1 and a vital sign detection method according to the first embodiment as described above, space occupied by the vital sign detection device 1 at an installation location can be reduced, the scope of selection of an installation location of the vital sign detection device 1 can be broadened, and the degree of freedom in design increases. In addition, the cost of the vital sign detection device 1 can be reduced.

As a result, according to the first embodiment, there can be provided the vital sign detection device 1 and a vital sign detection method with which noise included in the vital signs of the subject 3 can be removed and only the vital signs of the subject 3 can always be accurately extracted even if the size and orientation of the subject 3 change, and the increase in the degree of freedom in design and cost reduction can be achieved.

Since the frequency band(s) of the radio wave A having the first directivity and the radio wave B having the second directivity are set to be the same in the first embodiment, there is no need to emit radio waves in a different frequency band dislike in a biological sensor in the related art. The vital sign detection device 1 and a vital sign detection method can therefore be easily designed. Accordingly, the vital signs of the subject 3 can be accurately extracted at a low cost. The frequency band(s) of the radio wave A having the first directivity and the radio wave B having the second directivity do not necessarily have to be the same and may be set to be different from each other.

FIG. 6 is a schematic side view of the seat 2 on which the subject 3 sits in a vehicle to which a vital sign detection device and a vital sign detection method according to a second embodiment of the present disclosure are applied. A vital sign detection device according to the second embodiment has the same configuration as the above vital sign detection device 1 according to the first embodiment except that a reflector 31 is disposed in the seat 2 in addition to components in the above vital sign detection device 1 according to the first embodiment. The reflector 31 is formed of a material such as a metal that easily reflects radio waves and reflects, to the reception unit 13, the radio wave B having the second directivity emitted from the irradiation unit 11.

It is considered that, in the cases where the radio wave B is applied to the subject 3 and the radio wave B is applied to the reflector 31, the same calculation result of the body surface displacement of the subject 3 is obtained from the radio wave B having the second directivity for the irradiation region where the vital signs of the subject 3 are less likely to appear. Accordingly, in a vital sign detection device according to the second embodiment, control processing for changing the directivity of a radio wave to be emitted from the irradiation unit 11 to the subject 3 to the second directivity with which radio wave B is applied to the irradiation region where the vital signs of the subject 3 are less likely to appear can be easily performed with certainty by causing the control unit 15 to control the scan unit 12 such that the radio wave B emitted from the irradiation unit 11 has a directivity with which the radio wave B is applied to the reflector 31. In S201 to S204 in FIG. 4, processing for determining candidates for the second directivity therefore becomes unnecessary. As a result, the vital sign extraction unit 16 can easily remove noise included in the vital signs of the subject 3 with certainty while the control processing of the directivity determination unit 14 is simplified.

In the first and second embodiments, the case has been described where an electromagnetic wave is applied from the irradiation unit 11 to the subject 3 in S101 in FIG. 3 when the first directivity and the second directivity of electromagnetic waves are determined and an electromagnetic wave is reapplied from the irradiation unit 11 to the subject 3 with determined directivities in S103 in FIG. 3 when the vital signs of the subject 3 are extracted. However, a configuration may be employed in which electromagnetic waves having a plurality of directivities are applied from the irradiation unit 11 to the subject 3 only once, the reception unit 13 receives electromagnetic waves having the multiple directivities reflected from the subject 3, the first directivity and the second directivity of electromagnetic waves are determined on the basis of the electromagnetic waves received, and the vital signs of the subject 3 are extracted from the electromagnetic waves having the determined respective directivities.

The vital sign detection device 1 according to the third embodiment has a configuration that includes the irradiation unit 11 for emitting an electromagnetic wave to the subject 3, the scan unit 12 for scanning the irradiation region of an electromagnetic wave to be applied to the subject 3 by changing the directivity of an electromagnetic wave to be emitted from the irradiation unit 11, the reception unit 13 for receiving a plurality of electromagnetic waves having different directivities that have hit against and been reflected from the subject 3, and the vital sign extraction unit 16 for extracting the vital signs of the subject 3 using the difference between the distance information of the subject 3 calculated on the basis of an electromagnetic wave that shows the vital signs of the subject 3 and has the highest signal intensity of a plurality of electromagnetic waves received by the reception unit 13 and the distance information of the subject 3 calculated on the basis of an electromagnetic wave that shows characteristics other than the vital signs of the subject 3, that is, noise such as a body movement and a vibration, and has the highest signal intensity of the multiple electromagnetic waves.

A vital sign detection method having the above configuration includes an electromagnetic wave scanning step of causing the scan unit 12 to scan an irradiation region of an electromagnetic wave for the subject 3 by changing the directivity of an electromagnetic wave to be emitted from the irradiation unit 11 to the subject 3, an electromagnetic wave reception step of causing the reception unit 13 to receive a plurality of electromagnetic waves having different directivities that have been subjected to scanning in the electromagnetic wave scanning step and that reflect off the subject 3, a vital sign extraction step of extracting the vital signs of the subject 3 using the difference between the distance information of the subject 3 calculated on the basis of an electromagnetic wave that shows the vital signs of the subject 3 and has the highest signal intensity of a plurality of electromagnetic waves received by the reception unit 13 and the distance information of the subject 3 calculated on the basis of an electromagnetic wave that shows characteristics other than the vital signs of the subject 3, that is, noise such as a body movement and a vibration, and has the highest signal intensity of the multiple electromagnetic waves, a comparison step of comparing the size of vital signs of the subject 3 extracted in the vital sign extraction step with a predetermined threshold value, and a vital sign re-extraction step of, when the size of vital signs of the subject 3 extracted in the vital sign extraction step does not exceed the predetermined threshold value as a result of comparison in the comparison step, repeatedly performing the electromagnetic wave scanning step, the electromagnetic wave reception step, and the vital sign extraction step.

With the vital sign detection device 1 and a vital sign detection method according to the third embodiment, electromagnetic waves to be emitted from the single irradiation unit 11 to the subject 3 are caused to have a plurality of directivities and the electromagnetic waves are emitted from the irradiation unit 11. The electromagnetic waves emitted from the irradiation unit 11 are received by the reception unit 13 as electromagnetic waves having a plurality of directivities. Noise included in the vital signs of the subject 3 is removed and the vital signs of the subject 3 are accurately extracted by causing the vital sign extraction unit 16 to take the difference between the distance information of the subject 3 calculated on the basis of an electromagnetic wave that shows the vital signs of the subject 3 and has the highest signal intensity of a plurality of electromagnetic waves received by the reception unit 13 and the distance information of the subject 3 calculated on the basis of an electromagnetic wave that shows characteristics other than the vital signs of the subject 3 and has the highest signal intensity of the multiple electromagnetic waves.

Even if the size and orientation of the subject 3 change, electromagnetic waves having a plurality of new directivities are received by the reception unit 13 by causing the scan unit 12 to change the directivities of electromagnetic waves to be emitted from the irradiation unit 11 to the subject 3. On the basis of the multiple new electromagnetic waves received by the reception unit 13, the vital sign extraction unit 16 takes the difference between the distance information of the subject 3 calculated on the basis of an electromagnetic wave that shows the vital signs of the subject 3 and has the highest signal intensity of the multiple new electromagnetic waves received by the reception unit 13 and the distance information of the subject 3 calculated on the basis of an electromagnetic wave that shows characteristics other than the vital signs of the subject 3 and has the highest signal intensity of the multiple electromagnetic waves. As a result, noise included in the vital signs of the subject 3 is removed, and the new vital signs of the subject 3 are accurately extracted. Thus, even if the size and orientation of the subject 3 change, the vital signs of the subject 3 are accurately extracted.

Since the vital signs of the subject 3 are accurately extracted using the single irradiation unit 11 as above also in the vital sign detection device 1 and a vital sign detection method according to the third embodiment, space occupied by the vital sign detection device 1 at an installation location can be reduced, the scope of selection of an installation location of the vital sign detection device 1 can be broadened, and the degree of freedom in design increases. In addition, the cost of the vital sign detection device 1 can also be reduced.

The above vital sign detection device 1 according to the third embodiment may further include the directivity determination unit 14 and the control unit 15. In the vital sign detection device 1 according to a fourth embodiment having this configuration, the directivity determination unit 14 determines the first directivity of an electromagnetic wave that shows the vital signs of the subject 3 and has the highest signal intensity of a plurality of electromagnetic waves received by the reception unit 13 and the second directivity of an electromagnetic wave that shows characteristics other than the vital signs of the subject 3 and has the highest signal intensity of the multiple electromagnetic waves from a result of reception of electromagnetic waves in the reception unit 13 and stores the determined first directivity and the determined second directivity in a storage unit. The control unit 15 performs electromagnetic wave scanning control upon the scan unit 12 when the directivity of an electromagnetic wave to be emitted from the irradiation unit 11 is changed. The vital sign extraction unit 16 reads the first directivity of an electromagnetic wave and the second directivity of an electromagnetic wave stored in the storage unit and extracts the vital signs of the subject 3 on the basis of the difference between information about the distance from the vital sign detection device 1 to the electromagnetic wave irradiation point of the subject 3 calculated on the basis of the read electromagnetic wave having the first directivity and information about the distance from the vital sign detection device 1 to the electromagnetic wave irradiation point of the subject 3 calculated on the basis of the read electromagnetic wave having the second directivity.

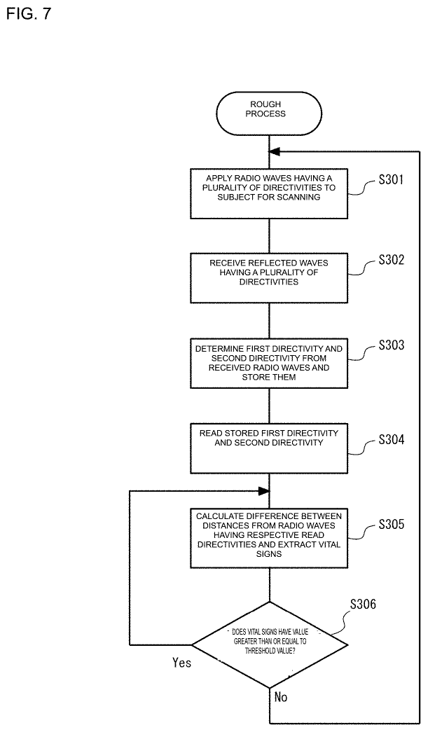

FIG. 7 is a flowchart schematically illustrating a vital sign detection method for which the vital sign detection device 1 according to the fourth embodiment of the present disclosure is used.

In this vital sign detection method, first, the scan unit 12 controls the control unit 15 to cause the scan unit 12 to scan the radio wave irradiation region of the subject 3 by changing the directivity of a radio wave to be applied from the irradiation unit 11 to the subject 3 in an electromagnetic wave scanning step in S301. Subsequently, in an electromagnetic wave reception step in S302, a plurality of electromagnetic waves having different directivities that have been subjected to scanning in the electromagnetic wave scanning step in S301 and reflected off the reception unit 13 are received by the reception unit 13. Subsequently, in a directivity determination step in S303, the directivity determination unit 14 determines, from a result of reception of electromagnetic waves in the reception unit 13, the first directivity of an electromagnetic wave that shows the vital signs of the subject 3 and has the highest signal intensity of a plurality of electromagnetic waves received by the reception unit 13 and the second directivity of an electromagnetic wave that shows characteristics other than the vital signs of the subject 3 and has the highest signal intensity of the multiple electromagnetic waves. The determined first directivity of an electromagnetic wave and the determined second directivity of an electromagnetic wave are stored in a storage unit by the directivity determination unit 14.

Subsequently, in a directivity reading step in S304, the vital sign extraction unit 16 reads the first directivity of an electromagnetic wave and the second directivity of an electromagnetic wave stored in the storage unit. In a vital sign extraction step in S305, the vital sign extraction unit 16 extracts the vital signs of the subject 3 on the basis of the difference between information about the distance from the vital sign detection device 1 to the electromagnetic wave irradiation point of the subject 3 calculated on the basis of the read electromagnetic wave having the first directivity and information about the distance from the vital sign detection device 1 to the electromagnetic wave irradiation point of the subject 3 calculated on the basis of the read electromagnetic wave having the second directivity. Subsequently, in a comparison step in S306, the size of the vital signs of the subject 3 extracted in the vital sign extraction step in S305 is compared with a predetermined threshold value. When the size of vital signs of the subject 3 is greater than or equal to the predetermined threshold value and a result of the compassion in S306 is Yes, the process returns to S305 and processing of S305 is repeated. On the other hand, when the size of vital signs of the subject 3 does not exceed the predetermined threshold value, the process returns to S301 and a vital sign re-extraction step is performed by repeating the radio wave scanning step in S301, the electromagnetic wave reception step in S302, the directivity determination step in S303, the directivity reading step in S304, and the vital sign extraction step in step S305.

With the vital sign detection device 1 and a vital sign detection method according to the fourth embodiment, noise included in the vital signs of the subject 3 is removed and the vital signs of the subject 3 can be accurately extracted by taking the difference between the distance information of the subject 3 calculated on the basis of an electromagnetic wave that shows the vital signs of the subject 3 and has the highest signal intensity of a plurality of electromagnetic waves received by the reception unit 13 and the distance information of the subject 3 calculated on the basis of an electromagnetic wave that shows characteristics other than the vital signs of the subject 3 and has the highest signal intensity of the multiple electromagnetic waves like the case where the vital sign detection device 1 and a vital sign detection method according to the third embodiment are used. Even if the size and orientation of the subject 3 change, new electromagnetic waves having a plurality of directivities are received by the reception unit 13 and the new vital signs of the subject 3 are accurately extracted by causing the control unit 15 to control the scan unit 12 to change the directivities of electromagnetic waves to be emitted from the irradiation unit 11 to the subject 3. Accordingly, even if the size and orientation of the subject 3 change, the vital signs of the subject 3 are accurately extracted. Since the vital signs of the subject 3 are accurately extracted using the single irradiation unit 11 as above also in the vital sign detection device 1 and a vital sign detection method according to the fourth embodiment, space occupied by the vital sign detection device 1 at an installation location can be reduced, the scope of selection of an installation location of the vital sign detection device 1 can be broadened, and the degree of freedom in design increases. In addition, the cost of the vital sign detection device 1 can also be reduced.

Since the frequency band(s) of the radio wave A having the first directivity and the radio wave B having the second directivity are set to be the same also in the third and fourth embodiments, there is no need to emit radio waves in different frequency bands dislike in a biological sensor in the related art. The vital sign detection device 1 and a vital sign detection method can therefore be easily designed. Accordingly, the vital signs of the subject 3 can be accurately extracted at a low cost.

Also in the third and fourth embodiments, the reflector 31 may be disposed in the seat 2 like in a vital sign detection device according to the second embodiment. With a vital sign detection device having the above configuration, noise included in the vital signs of the subject 3 can be easily removed by the vital sign extraction unit 16 with certainty by causing the radio wave B emitted from the irradiation unit 11 to have a directivity with which the radio wave B is applied to the reflector 31.

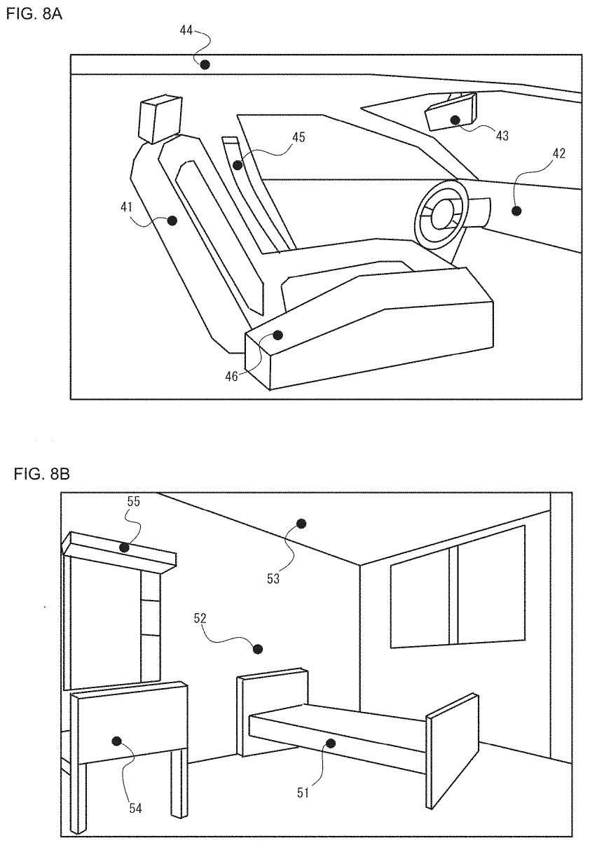

Although the case has been described in the above respective embodiments where the vital sign detection device 1 is disposed in a seat 41 in the interior of a car as illustrated in FIG. 8A, the vital sign detection device 1 may be disposed on/in, for example, a dashboard 42, a room mirror 43, a ceiling 44 in the interior of a vehicle, a seatbelt 45, or a glove box 46.

### INDUSTRIAL APPLICABILITY

Although the case has been described in the above respective embodiments where the present disclosure is applied to a driver monitoring system in a car, the present disclosure may be applied to a driver monitoring system in a vehicle, such as a plane or a train, by disposing a vital sign detection device according to an embodiment in a driver's seat in, for example, a plane or a train. As illustrated in FIG. 8B, the present disclosure may also be applied to watching over, for example, a patient by disposing a vital sign detection device according to the present disclosure in/on, for example, a bed 51, a wall 52, a ceiling 53, a chair 54, or a light 55 in a medical facility and causing the vital sign detection device to detect the vital signs of the patient.

### REFERENCE SIGNS LIST

1 vital sign detection device
2 seat
3 subject
11 irradiation unit
12 scan unit
13 reception unit
14 directivity determination unit
15 control unit
16 vital sign extraction unit
31 reflector
A radio wave having first directivity
B radio wave having second directivity

1. A vital sign detection device comprising:
a transmitter configured to emit electromagnetic waves toward a subject;
a scan controller configured to change a directivity of transmissions from the transmitter so as to scan an irradiation region of the subject with the electromagnetic waves;
a receiver configured to receive a plurality of returned electromagnetic waves with different directivities after having reflected off the subject; and
a vital sign extraction circuitry configured to extract vital signs of the subject using a difference between distance information of the subject calculated based on a particular electromagnetic wave that shows vital signs of the subject and has a highest signal intensity of the plurality of the electromagnetic waves received by the receiver and distance information of the subject calculated based on another electromagnetic wave that shows characteristics other than vital signs of the subject and has a highest signal intensity of the plurality of electromagnetic waves received by the receiver that show characteristics other than the vital signs of the subject.

2. The vital sign detection device according to claim 1, further comprising:
directivity determination circuitry configured to
determine, from electromagnetic waves received by the receiver, a first directivity of the particular electromagnetic wave that shows vital signs of the subject and has the highest signal intensity of a plurality of electromagnetic waves received by the receiver and a second directivity of the electromagnetic wave that shows characteristics other than vital signs of the subject and has the highest signal intensity of the plurality of electromagnetic waves, and
store the first directivity the second directivity in a storage unit, wherein
the scan controller includes a processing circuitry configured to a control directional scanning of transmissions of the transmitter, and
the vital sign extraction circuitry is configured to read the first directivity and the second directivity from the storage unit and extract vital signs of the subject based on a difference between information about a distance from the vital sign detection device to an electromagnetic wave irradiation point of the subject calculated based on the particular electromagnetic wave associated with the first directivity and information about a distance from the vital sign detection device to an electromagnetic wave irradiation point of the subject calculated based on the another electromagnetic wave associated with the second directivity.

3. The vital sign detection device according to claim 2, wherein frequency bands of the particular electromagnetic wave having the first directivity and the another electromagnetic wave having the second directivity are the same.

4. The vital sign detection device according to claim 2, further comprising:
a reflector configured to reflect to the receiver the another electromagnetic wave having the second directivity emitted from the transmitter.

5. A vehicle comprising:
a seat; and
the vital sign detection device according to claim 1 that is disposed in the seat.

6. A vehicle comprising:
a seat; and
the vital sign detection device according to claim 2 that is disposed in the seat.

7. A vital sign detection device comprising:
a transmitter configured to transmit electromagnetic waves toward a subject;
a scan controller configured to change a directivity of transmissions from the transmitter so as to scan an irradiation region of the subject with the electromagnetic waves;
a receiver configured to receive returned electromagnetic waves that have reflected off the subject;
directivity determination circuitry configured to determine, from returned energy received by the receiver, a first directivity of electromagnetic waves applied to the irradiation region where vital signs of the subject easily appear and a second directivity of electromagnetic waves applied to the irradiation region where vital signs of the subject are less likely to appear;
a scan controller configured to change a directivity of transmissions from the transmitter so as to scan the irradiation region of the subject with transmissions from the transmitter; and
vital sign extraction circuitry configured to extract vital signs of the subject based on a difference between information about a distance from the vital sign detection device to the subject calculated based on a particular electromagnetic wave having the first directivity that has been reflected from the subject and received by the receiver and information about a distance from the vital sign detection device to the subject calculated based on another electromagnetic wave having the second directivity that has been reflected off the subject and received by the receiver.

8. The vital sign detection device according to claim 7, wherein the receiver is configured to calculate a difference between information about a distance from the vital sign detection device to an irradiation point of the subject calculated based on the particular electromagnetic wave having the first directivity and information about another distance from the vital sign detection device to another irradiation point of the subject calculated based on the another electromagnetic wave having the second directivity that has been reflected from the subject and received by the receiver.

9. The vital sign detection device according to claim 7, wherein the directivity determination circuitry is further configured to
determine an irradiation direction of electromagnetic waves that are a candidate for each of the first directivity and the second directivity and set an irradiation direction from the transmitter to the determined irradiation direction accordingly, and
determine the first directivity and the second directivity and set directivities of transmissions from the transmitter to the determined first directivity and the determined second directivity.

10. The vital sign detection device according to claim 8, wherein the directivity determination circuitry is further configured to
determine an irradiation direction of electromagnetic waves that are a candidate for each of the first directivity and the second directivity and set an irradiation direction from the transmitter to the determined irradiation direction, and
determine the first directivity and the second directivity and set directivities of transmissions from the transmitter to the determined first directivity and the determined second directivity.

11. The vital sign detection device according to claim 9, wherein the directivity determination circuitry is further configured to determine, for each candidate, an irradiation range from the transmitter to the subject and a size of a scan angle at which scanning is performed in the irradiation range, and
the scan controller is configured to control the transmitter to emit transmissions in the determined irradiation range at the determined size of a scan angle.

12. The vital sign detection device according to claim 10, wherein the directivity determination circuitry is further configured to determine, for each candidate, an irradiation range from the transmitter to the subject and a size of a scan angle at which scanning is performed in the irradiation range, and
the scan controller is configured to control the transmitter to emit transmissions in the determined irradiation range at the determined size of a scan angle.

13. The vital sign detection device according to claim 9, wherein the directivity determination circuitry is configured to
compare a plurality of reception waveforms that have been determined as candidates for the first directivity by the candidate determination portion with a model waveform of an electromagnetic wave having the first directivity prepared in advance,
calculate a degree of similarity between each of the reception waveforms and the model waveform, and
determine the first directivity.

14. The vital sign detection device according to claim 10, wherein the directivity determination circuitry is configured to
compare a plurality of reception waveforms that have been determined as candidates for the first directivity by the candidate determination portion with a model waveform of an electromagnetic wave having the first directivity prepared in advance,
calculate a degree of similarity between each of the reception waveforms and the model waveform, and
determine the first directivity.

15. The vital sign detection device according to claim 8, wherein frequency bands for transmissions having the first directivity and the second directivity are the same.

16. The vital sign detection device according to claim 8, further comprising:
a reflector configured to reflect to the receiver transmissions at the second directivity that are transmitted from the transmitter.

17. A vehicle comprising:
a seat; and
the vital sign detection device according to claim 7 that is disposed in the seat.

18. A vital sign detection method comprising:
directing with a scan controller an electromagnetic wave emitted from a transmitter to scan an irradiation region of a subject by changing a directivity of transmissions from the transmitter;
receiving a plurality of reflected electromagnetic waves having different directivities after reflection off the subject;
extracting, with a processor, vital signs of the subject using a difference between distance information of the subject calculated based on a particular electromagnetic wave that shows vital signs of the subject and has the highest signal intensity of a plurality of electromagnetic waves received by the receiver and distance information of the subject calculated based on another electromagnetic wave that shows characteristics other than vital signs of the subject and has the highest signal intensity of the plurality of electromagnetic waves;
comparing a size of vital signs of the subject extracted in the extracting with a predetermined threshold value; and
performing re-extraction under a condition the size of vital signs of the subject extracted in the vital sign extraction step does not exceed the predetermined threshold value as a result of the comparing, and repeatedly performing the directing, receiving, and extracting.

19. The vital sign detection method of claim 18, wherein at least the directing and receiving are performed inside of a vehicle while the subject is disposed in a seat of the vehicle.

20. A non-transitory computer readable storage device having computer readable instructions stored therein that when executed by a processor cause the processor to perform at least the comparing and the extracting of claim 18.
